(19) United States
(12) Patent Application Publication
(10) Pub. No.: US 2008/0182823 A1
(43) Pub. Date: Jul. 31, 2008

(54) POLYMER-LINKED-BIOPHOSPHONATE INHALANT FORMULATIONS AND METHODS FOR USING THE SAME

(76) Inventors: Hidesmasa Katsumi, San Jose, CA (US); Akira Yamamoto, San Jose, CA (US); Maria Nakatani, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/963,666

(22) Filed: Dec. 21, 2007

Related U.S. Application Data
(60) Provisional application No. 60/897,553, filed on Jan. 26, 2007.

(51) Int. Cl. A61K 31/663; A61P 43/00

(52) U.S. Cl. 514/108

(57) ABSTRACT

The present invention provides for methods of administering a bisphosphonate active agent to a subject in need thereof. Aspects of the invention include administering the bisphosphonate active agent to the subject by a pulmonary route, where the bisphosphonate active agent is bonded, either directly or through an intervening linking group, to a non-peptide polymer, such that the bisphosphonate active agent is a polymer-linked-bisphosphonate active agent. Also provided are compositions for use in practicing methods according to embodiments of the invention. Methods and compositions according to embodiments of the invention find use in a variety of different applications, including but not limited to, the treatment of bone adsorption disease conditions.

### CROSS-REFERENCE TO RELATED APPLICATIONS

Pursuant to 35 U.S.C. §119(e), this application claims priority to the filing date of U.S. Provisional Patent Application Ser. No. 60/897,553 filed Jan. 26, 2007; the disclosure of which is herein incorporated by reference.

### INTRODUCTION

Bisphosphonates and their pharmacologically acceptable salts find use in a variety of different applications. For example, bisphosphonates have been employed as bone absorption inhibitors in treating patients suffering from osteoporosis, Paget's disease and cancer.

In the past, bisphosphonates have been administrated orally and intravenously. However, there are disadvantages associated with the oral and intravenous administration of bisphosphonates. For example, the bioavailability of a bisphosphonate following oral administration can be very low. Furthermore, bisphosphonates can be irritating to the gastrointestinal tract. In addition, patient compliance can be problematic as patients are typically prevented from lying down following oral administration.

Intravenous administration of bisphosphonates, while overcoming some of the disadvantages of oral administration, is not entirely satisfactory. For example, because rapid intravenous administration of bisphosphonates may cause renal complications, intravenous bisphosphonate administration generally takes a long period of time. Lichtenberger et al. (Dig. Dis. Sci. 45(9):1792-1801, 2000) have shown that the administration of alendronate, pamidronate, or risedronate cause antral mucosal injury in rat models.

Because of the above disadvantages of oral and intravenous bisphosphonate administration, inhalation administration of bisphosphonates has been proposed. See e.g., U.S. Pat. No. 6,743,414. However, inhalation administration of bisphosphonates can be damaging to the pulmonary mucosal tissue.

### SUMMARY

The present invention provides for methods of administering a bisphosphonate active agent to a subject in need thereof. Aspects of the invention include administering the bisphosphonate active agent to the subject by a pulmonary route, where the bisphosphonate active agent is bonded, either directly or through an intervening linking group, to a non-peptide polymer, such that the bisphosphonate active agent is a polymer-linked-bisphosphonate active agent. Also provided are compositions for use in practicing methods according to embodiments of the invention. Methods and compositions according to embodiments of the invention find use in a variety of different applications, including but not limited to, the treatment of bone adsorption disease conditions.

### BRIEF DESCRIPTION OF THE DRAWINGS

FIG. 1 shows the calculation of D % derived from plasma calcium-time profile after intrapulmonary administration of alendronate in rates.

FIG. 2 shows plasma concentration of calcium after intrapulmonary administration of PEG-alendronate (PEG-ALN) in rats.

FIG. 3 shows Pharmacological activity of PEG-alendronate (PEG-ALN) after its intrapulmonary administration in rats.

FIG. 4 shows LDH activity and total protein level in bronchoalveolar lavage fluid (BALF) at 4 h after intrapulmonary administration of PEG-alendronate (PEG-ALN) in rats.

FIG. 5 shows determination of molecular weight of PEG(500)-alendronate (PEG(500)-ALN) by TOF-MASS

### DEFINITIONS

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope.

“Alkyl” refers to monovalent saturated aliphatic hydrocarbyl groups particularly having up to 30 carbon atoms, or up to 10 carbon atoms, up to 9 carbon atoms, up to 8 carbon atoms, or up to 3 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “alkyl” also includes “cycloalkyls” as defined herein.

“Cycloalkyl” refers to cyclic hydrocarbyl groups having from 3 to about 30 carbon atoms, or from 3 to about 10 carbon atoms, and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like. The term “cycloalkyl” also includes “heterocycloalkyls” as defined herein.

“Heterocycloalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of such heterocyclic non-aromatic rings include, but are not limited to, aziridinyl, azetidinyl, piperazinyl, and piperidinyl.

“Heteroaryl” refers to a stable heterocyclic aromatic ring and fused rings containing one or more heteroatoms independently selected from N, O and S. A fused heterocyclic ring system may include carbocyclic rings and need only include one heterocyclic ring. Examples of such heterocyclic aromatic rings include, but are not limited to, pyridine, pyrimidine, and pyrazinyl.

“Aryl” refers to a monovalent aromatic hydrocarbon group derived by the removal of one hydrogen atom from a single carbon atom of a parent aromatic ring system. Typical aryl groups include, but are not limited to, groups derived from benzene, ethylbenzene, mesitylene, toluene, xylene, aniline, chlorobenzene, nitrobenzene, and the like. The term “aryl” also includes “heteroaryl” as defined herein.

“Halogen” refers to fluoro, chloro, bromo and iodo. In some embodiments, the halogen is fluoro or chloro.

“Substituted” refers to a group in which one or more hydrogen atoms are each independently replaced with the same or different substituent(s). “Substituted” groups particularly refer to groups having 1 or more substituents, for instance from 1 to 5 substituents, and particularly from 1 to 3 substituents, selected from the group consisting of amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryl, substituted thioaryl, thioketo, thiol, alkyl-S(O)—, aryl-S(O)—, alkyl-S(O)₂— and aryl-S(O)₂.

### DETAILED DESCRIPTION

The present invention provides for methods of administering a bisphosphonate active agent to a subject in need thereof. Aspects of the invention include administering the bisphosphonate active agent to the subject by a pulmonary route, where the bisphosphonate active agent is bonded, either directly or through an intervening linking group, to a non-peptide polymer, such that the bisphosphonate active agent is a polymer-linked-bisphosphonate active agent. Also provided are compositions for use in practicing methods according to embodiments of the invention. Methods and compositions according to embodiments of the invention find use in a variety of different applications, including but not limited to, the treatment of bone adsorption disease conditions.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

In further describing the subject invention, the subject methods are described first in greater detail, followed by a review of the various compositions, e.g., formulations and kits, that may find use in the subject methods, as well as a discussion of various representative applications in which the subject methods and compositions find use.

### METHODS

Aspects of the invention include methods of administering a bisphosphonate active agent to a subject. Embodiments of the invention include administering the bisphosphonate active in a form where the active agent is bonded, either directly or through a linking group, to an irritation-reducing polymer, such that the active agent may be viewed as a polymer-linked-bisphosphonate active agent. The subject may be in need thereof, e.g., for the treatment of a disease or condition treatable by a bisphosphonate active agent (as described in greater detail below). Aspects of the subject methods include administering a polymer-linked-bisphosphonate active agent to a subject, e.g., via a pulmonary route.

### Polymer-Linked-Bisphosphonate Active Agent

Aspects of the methods include administering the bishosphonate active agent to a subject, where the active agent is a polymer-linked bisphosphonate active agent as summarized above.

Polymer-linked-bisphosphonate active agents of interest are polymer modified bisphosphonate compounds, where the bisphosphonate compounds are capable of inhibiting the resorption of bone. Bisphosphonate compounds are also known as diphosphonates or bisphosphonic acid.

The polymer-linked-bisphosphonate active agents employed in embodiments of the methods of the invention may have a high affinity to bone tissue. In some embodiments, the polymer-linked-bisphosphonate active agent metabolizes in a cell into compounds that compete with adenosine triphosphate (ATP) in the cellular energy metabolism. In some embodiments, the polymer-linked-bisphosphonate active agent binds the farynesyl disphosphate synthase (FPPS) enzyme and inhibits the enzymatic activity of FPPS. FPPS is an enzyme involved in the 3-hydroxy-3-methylglutaryl-CoA (HMG-CoA) reductase pathway (or mevalonate pathway).

Whether or not a given polymer-linked-bisphosphonate active agent is suitable for use according to the present invention can be readily determined using assays employed in the experimental section, below. In certain embodiments, a polymer-linked-bisphosphonate active agent is suitable for use in the subject methods if it exhibits desired activity as determined using the in situ trans-pulmonary absorption test described in the experimental section below.

Polymer-linked bisphosphonate compounds employed in embodiments of the invention may include an irritation reducing polymer that is bonded, either directly or through a linking group, to a bisphosphonate active agent. The irritation reducing polymer is one that provides for about a 5% or more, such as about a 10% or more, including about a 25% or more reduction in irritation as determined using the assays described in the experimental section below, as compared to a control. In certain embodiments, the amount of irration reduction provided by the polymer component of the conjugated bisphosphonate active agent is about 50% or more, such as about 75% or more, including about 90% or more.

Polymer-linked-bisphosphonate active agents of interest include compounds of the following structure:

PM-L-BP;

wherein:

PM is a linear or branched water-soluble and non-peptide polymer having at least one terminus, wherein the terminus is covalently bonded to the L;

L is a bond or linker group; and

BP is a bisphosphonate group.

The linear or branched water-soluble and non-peptide polymer is a substantially non-immunogenic polymer, such as a poly(alkylene glycol), such as poly(ethylene glycol) (PEG). Other related polymers are also suitable for use in the practice of this invention and that the use of the term PEG or poly(ethylene glycol) is intended to be inclusive and not exclusive in this respect. In some embodiments, the polymer has from 2 to about 300 termini.

In some embodiments, the polymer is clear, colorless, odorless, soluble in water, stable to heat, inert to many chemical agents, does not hydrolyze or deteriorate, and is nontoxic. In some embodiments, the polymer is biocompatible, which is to say that the polymer is capable of coexistence with living tissues or organisms without causing harm. In some embodiments, the polymer is non-immunogenic, which is to say that the polymer does not produce an immune response in the body. In some embodiments, the polymer is a PEG comprising the formula R^{a}—(CH₂CH₂O)ₘ—, where m is from about 3 to about 4000, or from about 3 to about 2000, and R^{a} is a hydrogen, —OH, CH₃—O—, CH₂CH₂—O—, CH₃CH₂CH₂—O— or CH₃—.

The polymer can be linear or branched. In some embodiments, a branched polymer has a central branch core moiety and a plurality of linear polymer chains linked to the central branch core. PEG includes branched forms that can be prepared by addition of ethylene oxide to various polyols, such as glycerol, pentaerythritol and sorbitol. The branched PEGs can be represented in general form as R^{b}(-PEG-OH)_{N} in which R^{b} represents the core moiety, such as glycerol or pentaerythritol, and n represents the number of arms and is from 2 to 300. In some embodiments, the PM is a linear or branched PEG.

Suitable polymers for the invention include, but are not limited to, poly(alkylene glycol), such as poly(ethylene glycol) (PEG) and poly(propylene glycol) (PPG), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, and copolymers, terpolymers, derivatives and mixtures thereof. The molecular weight of each chain of the polymer can vary in the range of from about 100 Da to about 100,000 Da, or from about 6,000 Da to about 80,000 Da. In some embodiments, the polymer further comprises R^{a} (as defined above) attached to all termini except the terminus that is bonded to the “bisphosphonate group”.

Suitable PEGs include, but are not limited to, PEG(100), PEG(200), PEG(300), PEG(400), PEG (500), PEG(600), PEG(1000), PEG(1500), PEG(2000), PEG(3000), PEG(3350), PEG(4000), PEG(5000), PEG(6000), PEG(8000), and PEG(10000), and methoxy and ethoxy derivatives thereof, and any PEG having a molecular size within and inclusive of any of the above indicated molecular weights.

In some embodiments, the polymer is a PEG comprising the formula R^{c}—(CH₂CH₂O)ₚ—, where p is from about 3 to about 4000, or from about 3 to about 2000, and R^{c} is a hydrogen, CH₃—O—, CH₂CH₂—O—, CH₃CH₂ CH₂—O— or CH₃—.

The polymer component may be synthesized using any convenient protocol or purchased from a commercial source, as desired. Suitable PEGs are commercially available from many sources, such as Sigma-Aldrich Corp. (St. Louis, Mo.).

Those of ordinary skill in the art will recognize that the foregoing list for substantially water soluble non-immunogenic polymer is by no means exhaustive and is merely illustrative, and that all polymeric materials having the qualities described above are contemplated.

The “linker” is a bond, the residue of a functional group used to attach the bisphosphonate group to the polymer selected from the group consisting of ketone linkages (e.g., diketone linkages), ester linkages, ether linkages, thio-ether linkages, amide linkages, amine linkages, urea linkages, or carbamate linkages. In some embodiments, the linker comprises an ketone linkage, e.g., a diketone linkage. In some embodiments, the linker is:

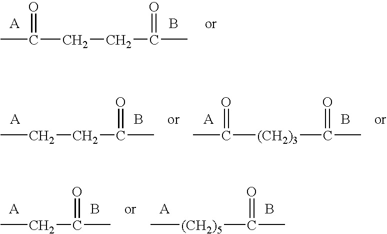

wherein bond A is attached to PM and bond B is attached to BP. When the linker is a functional group used to attach the bisphosphonate group to the polymer, it can be a hydrolytically stable linkage selected from the group consisting of ether linkages, thio-ether linkages, amide linkages, amine linkages, urea linkages, and carbamate linkages.

The “bisphosphonate group” is a compound that is characterized by two carbon-phosphorous bonds, P—C—P. Suitable bisphosphonate groups include compounds of formula (I):

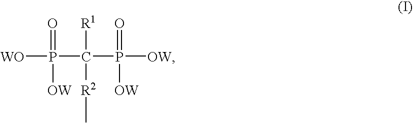

wherein R¹ and R² are independently selected from the group consisting of hydrogen, —OH, halogen, aryl, substituted aryl, pyridyl, furanyl, pyrrolidinyl, imidazonyl, C₁-C₃₀ alkyl, C₁-C₃₀ substituted alkyl, NH₂, NHR³, NR³₂, SH, and SR³, where R³ is C₁-C₃₀ alkyl, C₁-C₁₀ alkoxy, aryl or substituted aryl, where each carbon atom of R² may be optionally replaced with a nitrogen or sulfur atom and R² has no more than 3 nitrogen or sulfur atoms in total; and W is selected from the group consisting of hydrogen, alkyl, substituted alkyl, aryl, substituted aryl, Na⁺, and K⁺; with the provisio that R² is not a hydrogen, —OH, halogen, NH₂, or SH.

In some embodiments, the bisphosphonate group is a compound of formula (II):

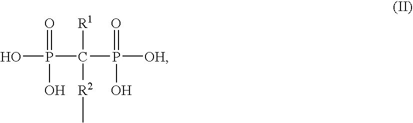

wherein R¹ and R² are as described above.

In certain embodiments, R² is selected from the group consisting of a substituted C₁-C₉ alkyl, unsubstituted C₁-C₉ alkyl, substituted C₁-C₉ cycloalkyl, unsubstituted C₁-C₉ cycloalkyl, substituted C₁-C₉ aryl, or unsubstituted C₁-C₉ aryl, wherein each carbon atom of R² may be optionally replaced with a nitrogen or sulfur atom and R² has no more than 2 nitrogen or sulfur atoms in total, wherein R² has no more than 8 carbon atoms.

In certain embodiments, R² is a C₁-C₈ alkyl, wherein the each carbon atom of R² may be optionally replaced with a nitrogen atom and the total number of nitrogen is R² is not more than 1, wherein the C₁-C₈ alkyl may be optionally substituted with an amino group.

In some embodiments, R¹ is —OH or fluorine and R² is a C₁-C₅ alkyl, which may optionally be substituted by a substituent such as amino groups and/or fluorine atoms.

In some embodiments, R¹ is —OH, R² is —NH(CH₂)_{q}—, where q is about 2 to about 6, and each W is hydrogen.

In some embodiments, R² is —CH₂—, —CH₂—CH₂—NH—, —(CH₂)₅—NH—,

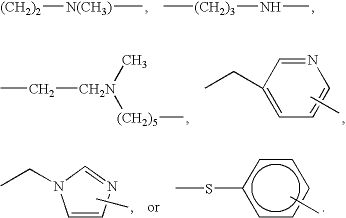

Specific “bisphosphonate groups” of interest include, but are not limited to: (4-amino-1-hydroxybutylidene)-bis-phosphonate or 4-amino-1-hydroxybutane-1,1-biphosphonic acid (alendronate); (Dichloromethylene)- bis-phosphonate (clodronate); (1-Hydroxyethylidene)-bis-phosphonate (etidronate); [1-Hydroxy-3-(methylpentylamino)propylidene] bis-phosphonate (ibandronate); [(Cycloheptylamino)-methylene] bis-phosphonate (incadronate); [1-Hydroxy-2-imidazo-(1,2-a)pyridine-3-ylethylidene] bis-phosphonate (minodronate); (6-amino-1-hydroxyhexylidene) bis-phosphonate (neridronate); [3-(Dimethylamino)-hydroxy-propylidene] bis-phosphonate (olpadronate); (3-Amino-1-hydroxypropylidene) bis-phosphonate (pamidronate); [1-Hydroxy-2-(3-pyridinyl)-ethylidine] bis-phosphonate (risedronate); [[4-Chlorophenyl)thio]-methylene] bis-phosphonate (tiludronate); [1-Hydrxy-2-(1H-imidazole-1-yl)ethylidene] bis-phosphonate (zoledronate); [(Cycloheptylamino)-methylene] bis-phosphonate (incadronate); [1-Hydroxy-2-imidazo-(1,2-a)pyridine-3-ylethylidene] bis-phosphonate (minodronate);5-amino-1-hydroxypentan-1,1-biphosphonic acid; 4-amino-1-hydroxybutan-1,1-biphosphonic acid; difluoro-methanbiphosphonic acid; and pharmacologically acceptable salts thereof.

Pharmacologically acceptable salts include, are not limited to, salts of alkali metal (e.g., sodium and potassium), salts of alkali earth metals (e.g., calcium), salts of inorganic acids (e.g., HCl), and salts of organic acids (e.g., citric acids and amino acids, such as lysine). In one embodiment, the bisphosphonate active agent is a salt of sodium.

In some embodiments of the invention, the polymer-linked-bisphosphonate active agent is a PEGylated bisphosphonate.

In some embodiments of the invention, the polymer-linked-bisphosphonate active agent is of the following structure:

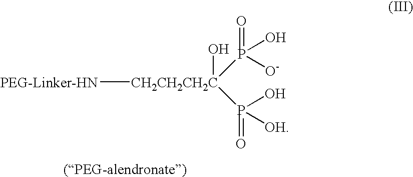

In certain embodiments of the invention, the polymer-linked-bisphosphonate active agent is of the following structure (IV) wherein the PEG and Linker are PEG(2000), —COCH₂CH₂CO—, respectively. (“PEG(2000)-alendronate”).

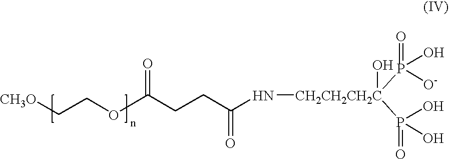

In certain embodiments of the invention, the polymer-linked-bisphosphonate active agent is of structure (V) wherein the PEG and Linker are PEG(500), —CH₂CH₂CO—, respectively. (“PEG(500)-alendronate”)

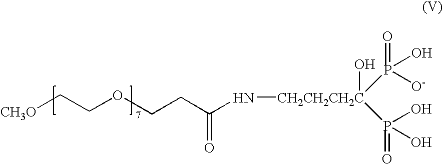

The polymer-linked-bisphosphonate active agent also includes the pharmaceutically acceptable salts, solvates, hydrates, and prodrug forms thereof, and stereoisomers thereof.

The scope of the present invention includes prodrugs of the polymer-linked-bisphosphonate active agent. Such prodrugs are in general functional derivatives of the compounds that are readily convertible in vivo into the required compounds. Thus, in the methods of the present invention, the term “administering” encompasses administering the compound specifically disclosed or with a compound which may not be specifically disclosed, but which converts to the specified compound in vivo after administration to the subject in need thereof. Conventional procedures for the selection and preparation of suitable prodrug derivatives are described, e.g., in Wermuth, “Designing Prodrugs and Bioprecursors” in Wermuth, ed. The Practice of Medicinal Chemistry, 2d Ed., pp. 561-586 (Academic Press 2003). Prodrugs include esters that hydrolyze in vivo (e.g., in the human body) to produce a compound described herein suitable for the present invention. Suitable ester groups include, without limitation, those derived from pharmaceutically acceptable, aliphatic carboxylic acids, particularly alkanoic, alkenoic, cycloalkanoic and alkanedioic acids. In some embodiments, each alkyl or alkenyl moiety has no more than 6 carbon atoms. Illustrative esters include formates, acetates, propionates, butyrates, acrylates, citrates, succinates, and ethylsuccinates.

The bisphosphonate group useful in the subject compositions include, but are not limited to those compounds described in U.S. Pat. Nos. 4,621,077; 5,183,815; 5,358,941; 5,462,932; 5,661,174; 5,681,590; 5,994,329; 6,015,801; 6,090,410; 6,225,294; 6,414,006; 6,482,411; and 6,743,414; the disclosures of which are herein incorporated by reference. Methods of synthesis of these bisphosphonate compounds are provided in these references.

Any convenient method of linking the polymer component to the bisphosphonate component may be employed. Methods of linking the polymer to the bisphosphonate group include those described in U.S. Pat. No. 6,436,386, the disclosure of which is herein incorporate by reference.

### Formulations and Adminstration

Also provided are pharmaceutical compositions containing the polymer-linked-bisphosphonate active agent employed in the subject methods. In certain embodiments, the polymer-linked-bisphosphonate active agent, e.g., in the form of a pharmaceutically acceptable salt, are formulated for pulmonary administration to a subject.

By way of illustration, the polymer-linked-bisphosphonate active agent can be admixed with conventional pharmaceutically acceptable carriers and excipients (i.e., vehicles) and used in forms suitable for pulmonary administration. Such suitable forms include aqueous solutions, suspensions, and the like. Such pharmaceutical compositions contain, in certain embodiments, from about 0.1 to about 90% by weight of the active compound, such as from about 1 to about 30% by weight of the active compound. The pharmaceutical compositions may contain common carriers and excipients, such as corn starch or gelatin, lactose, dextrose, sucrose, mannitol, sodium chloride, and alginic acid. The pharmaceutically acceptable excipients include, for example, any suitable vehicles, adjuvants, carriers or diluents, and are readily available to the public. The pharmaceutical compositions of the present invention may further contain other active agents as are well known in the art.

A liquid composition may be present as a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder.

One skilled in the art will appreciate that a variety of suitable methods of administering a formulation of the present invention to a subject, are available, and, although more than one route can be used to administer a particular formulation, a particular route can provide a more immediate and more effective reaction than another route. Pharmaceutically acceptable excipients may be employed as desired. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting.

The subject formulations of the present invention can be made into aerosol formulations to be administered via inhalation. These aerosol formulations (i.e., inhalant formulations) can be placed into pressurized acceptable propellants, such as dichlorodifluoromethane, propane, nitrogen, and the like. They may also be formulated as pharmaceuticals for non-pressured preparations, such as for use in a nebulizer or an atomizer.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Those of skill in the art will readily appreciate that dose levels can vary as a function of the specific compound, the nature of the delivery vehicle, and the like. Suitable dosages for a given compound are readily determinable by those of skill in the art by a variety of means.

The dose administered to an animal, particularly a human, in the context of the present invention should be sufficient to effect a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to bone adsorption inhibiting agents that are known to reduce bone loss due to bone adsorption.

Optionally, the pharmaceutical composition may contain other pharmaceutically acceptable components, such a buffers, surfactants, viscosity modifying agents, preservatives and the like. Each of these components is well-known in the art. See, e.g., U.S. Pat. No. 5,985,310, the disclosure of which is herein incorporated by reference. Other components suitable for use in the formulations of the present invention can be found in Remington's Pharmaceutical Sciences, Mace Publishing Company, Philadelphia, Pa., 17th ed. (1985).

In certain embodiments, the formulations of the present invention are administered to the host by a pulmonary route. In some embodiments, the pulmonary route of administration is in an inhalation dosage form directly into the respiratory tract, or directly to the respiratory airway, trachea, bronchi, bronchioles, lungs, alveolar ducts, alveolar sacs, and/or alveoli. The formulations may be administered by any convenient method, such as but not limited to: inhalers, metered dose, nebulizers, atomizers, breath activated or powder. The methods of the present invention also include administrating the formulations directly into the nasal cavity or oral cavity of the host with a dropper, pipette or kanule.

In certain embodiments, the formulation is in a powder form. The agents may be used as a powder with a particle size ranging from about 1 to about 10 μm, such as from about 2 to about 8 μm. For pharmaceutical purposes the particle size of the powder may be no greater than about 100 μm diameter. In certain embodiments, the particle size of the finely-divided solid powder is about 25 μm or less, such as about 10 μm or less in diameter. The particle size of the powder for inhalation therapy may range from about 2 to about 10 μm.

The concentration of medicament depends upon the desired dosage, and in certain embodiments ranges from about 0.01 to 5% by weight. A dosage in inhalation form may include 50-100 micrograms per day and administration of the inhalant composition may be on a once a day or once a week schedule. However the precise therapeutic dosage amount will depend on the age, size, sex and condition of the subject, the nature and severity of the disorder, and other such factors. An ordinarily skilled physician or clinician can readily determine and prescribe the effective amount of the drug required for a particular patient.

In some embodiments, the formulations are powdered aerosol formulations which include the active agents suspended or dispersed in a propellant or a propellant and solvent. The propellant generally comprises a mixture of liquefied chlorofluorocarbons (CFCs) which are selected to provide the desired vapor pressure and stability of the formulation. Propellants 11, 12 and 114 are the most widely used propellants in aerosol formulations for inhalation administration. Other commonly used propellants include Propellants 113, 142b, 152a 124, and dimethyl ether, which are commercially available from DuPont FluroChemicals (Wilmington, Del.). The compound 1,1,1,2-tetrafluoroethane is also a commonly used propellant for medicinal aerosol formulations. The propellant comprises 40 to 90% by weight of the total inhalation composition.

The inhalation composition may also contain dispersing agents and solvents, such as methylene chloride, ethanol or phosphate buffer solution (PBS). Surfactants have also been used as dispersing agents. Such agents include sorbitan tiroleate, oleyl alcohol, oleic acid, lecithin or oils derived from natural sources, such as, corn oil, olive oil, cotton seed oil and sunflower seed oil are useful in keeping the suspended particles form agglomerating. The surface active agents are generally present in amounts not exceeding 5% by weight of the total formulation. They maybe present in the weight ratio 1:100 to 10:1 surface active agent to bisphosphonate active agent, but the surface active agent may exceed this weight ratio in cases where the drug concentration in the formulation is very low.

The inhalation formulation of the present invention can be delivered in any convenient inhalation device, where the device may include a nebulizer or an atomizer.

In the methods and compositions of the present invention, the pharmaceutical composition may be administered in admixture with suitable pharmaceutical diluents, excipients or carriers. Moreover, when desired or necessary, suitable excipients, lubricants, disintegrating agents and coloring agents can also be incorporated into the mixture of active ingredient(s) and inert carrier materials. Suitable excipients may include starch, gelatin, natural sugars such as glucose, anhydrous lactose, free-flow lactose, beta-lactose, and corn sweeteners, natural and synthetic gums, such as acacia, tragacanth or sodium alginate, carboxymethyl cellulose, polyethylene glycol, waxes, cross carmallose sodium, and the like. Lubricants used in these dosage forms include sodium oleate, sodium stearate, magnesium stearate, sodium benzoate, sodium acetate, sodium chloride and the like.

In some embodiments, the pharmaceutical composition is a powder formulation comprising a polymer-linked-bisphosphonate active agent, or pharmacologically acceptable salt thereof. In certain embodiments, the pharmaceutical composition further comprises one or more excipients, such as a plasticizer, lubricant, binder, disintegrator, stabilizer, or masking agent. In certain embodiments, the surface of the particles of the powder formulation are coated with a suitable coating agent. Suitable coating agents include, but are limited to, enteric polymers, such as sureteric, cellulose acetate phthalate, methacrylic acid copolymer, hydroxypropyl methylcellulose phthalate, aquacoat ECD 30, shellac and zein. In certain embodiments, the pharmaceutical composition further comprises a lubricant, such as isopropyl myristate, light mineral oil or other substances which provide slippage between particles of the compound as well as lubrication for component parts of the valve of the inhalation device.

In some embodiments, the pharmaceutical composition is a solution or suspension formulation comprising a bisphosphonate active agent, or pharmacologically acceptable salt thereof. In certain embodiments, the solution or suspension formulation comprises the agents dissolved or suspended in water. In certain embodiments, the solution or suspension formulation further comprises one or more co-solvents, such as, ethanol, propylene glycol, or polyethylene glycol. In certain embodiments, the solution or suspension formulation further comprises one or more preservatives, solubilizers, buffering agents, isotonizers, surfactants, absorption enhancers, or viscosity enhancers. In certain embodiments, when the pharmaceutical composition is a suspension formulation and further comprises a suspending agent.

### Utility

The subject methods find use in a variety of applications, where in certain applications the methods are methods of modulating at least one cellular function, such as inhibiting bone reabsorption. The subject methods find use in treating, reducing the probably of, or preventing bone adsorption, loss of bone mass, osteoporosis, osteopenia, urolithiasis, hypercalcemia, Paget's disease (or osteitis deformans), bone metastasis, multiple myeloma, neoplastic bone lesions, and other conditions that cause or increase the risk of bone fragility. In some embodiments of the invention, the subject methods are also useful for reducing the probability or risk of non-vertebral fractures. In certain embodiments, the subject in need of the polymer-linked-bisphosphonate active agent is osteoroporotic or postmenopausal, or both. In certain embodiments, the subject is a woman who is osteoroporotic or postmenopausal, or both. In certain embodiments, the subject is a human juvenile with osteogenesis imperfecta.

In this respect, the subject methods and composition find use in known applications of bisphosphonate, such as in treating diseases or disorders that are capable of being treated using bisphosphonate. Use of the subject compositions of the present invention is of particular utility in, for example, in the treatment of diseases and disorders including but not limited to osteoporosis, osteopenia, urolithiasis, hypercalcemia, Paget's disease (or osteitis deformans), bone metastasis, multiple myeloma, neoplastic bone lesions, and other conditions that cause or increase the risk of bone fragility. In these capacities, use of the present inventive compositions will result in a reduced unwanted toxicity while a retention of desired bisphosphonate activity.

As such, the subject methods and compositions find use in therapeutic applications in which bisphosphonate administration is indicated. A representative therapeutic application is the treatment of bone disease conditions, e.g., osteoporosis and related conditions characterized by bone adsorption and loss of bone mass.

By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans. In some embodiments, the hosts are women.

The subject methods find use in, among other applications, the treatment of bone disease conditions, including osteoporosis conditions. In such applications, an effective amount of the polymer-linked-bisphosphonate active agent is administered to the subject in need thereof. Treatment is used broadly as defined above, e.g., to include at least amelioration in one or more of the symptoms of the disease, as well as a complete cessation thereof, as well as a reversal and/or complete removal of the disease condition, e.g., cure.

The dose administered to an animal, particularly a human, in the context of the present invention should be sufficient to effect a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to agents that are known to inhibit bone adsorption, particularly unmodified bisphosphonate. A suitable dosage is an amount which results in the inhibition of bone adsorption, without significant side effects. In proper doses and with suitable administration of certain compounds, the present invention provides for a wide range of intracellular effects, e.g., from partial inhibition to essentially complete inhibition of bone adsorption.

Individuals may be diagnosed as being in need of the subject methods using any convenient protocol, and are generally known to be in need of the subject methods, e.g., they are suffering from a target disease condition or have been determined to be at risk for suffering from a target disease condition, prior to practicing the subject methods.

Particular applications in which the subject methods and compositions find use include those described in U.S. Pat. Nos. 4,621,077; 5,183,815; 5,358,941; 5,462,932; 5,661,174; 5,681,590; 5,994,329; 6,015,801; 6,090,410; 6,225,294; 6,414,006; 6,482,411; and 6,743,414; the disclosures of which are herein incorporated by reference.

### Kits & Systems

Also provided are kits that find use in practicing the subject methods, as described above. For example, kits and systems for practicing the subject methods include a pharmaceutical formulation comprising the polymer-linked-bisphosphonate active agent. As such, in certain embodiments the kits may include a pharmaceutical composition, present as one or more unit dosages, where the composition includes the polymer-linked-bisphosphonate active agent.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The term “system” as employed herein refers to a collection of material including a composition comprising the polymer-linked-bisphosphonate active agent for the purpose of practicing the subject methods.

The following examples further illustrate the present invention and should not be construed as in any way limiting its scope.

### Experimental

### Experiment Materials

### Reagents

Alendronate (Toronto Research Chemicals Inc.) was provided by Teikoku Pharma USA, Inc. Methoxypolyethylene glycolyl N-succinimidyl succinate (SUNBRIGHT ME-020CS©, amino group reactive activation PEG (2000)) was purchased from NOF CORPORATION. Methoxypolyethylene glycolyl N-succinimidyl succinate (Methyl-PEO8-NHS Ester©, amino group reactive activation PEG (500))was purchased from PIERCE.

### Animals

A Wistar male rat was purchased from Shizuoka Agricultural Cooperative Association for Laboratory Animals. All the animal tests were conducted in accordance with the guideline established by the Animal Ethics Committee at Kyoto Pharmaceutical University.

### Experiment Methods

### Dosing Solution

38.5 μmol/ml (12.5 mg/ml) of Alendronate was prepared for transpulmonary administration by using the isotonic phosphate buffer solution (PBS) with the pH of 7.4. 38.5 μmol/ml (12.5 mg alendronate/ml) of PEG (2000)-alendronate and 38.5 μmol/ml (12.5 mg alendronate/ml) of PEG(500)-alendronate was prepared for transpulmonary administration by using the isotonic phosphate buffer solution (PBS) with the pH of 7.4.

### Transpulmonary Administration

A transpulmonary absorption test was conducted in the following method based on the method disclosed by Enna & Schanker (Am. J Physiol. 222(2):409-414, 1972; Am. J Physiol. 223(5):1227-1231, 1972) A Wister male rat weighing 250 to 300 g was used in the test. Under pentobarbital anesthesia, the center of the neck of the rat was cut open to expose the bronchial tract. A 2.5 cm long polyethylene tube (ID 1.5 mm, OD 2.3 cm) was inserted from the thyroid cartilage between the 4^{th} and 5^{th} bronchial cartilage rings to a 0.6 cm depth, and the open skin was then stitched up. A 100 μl microsyringe (Microliter, no. 710, Hamilton Colo.) was filled with 100 μl of the dosing solution. The rat was placed at 80°. The tip of the microsyringe was inserted at 1 to 2 mm up into the bronchial tract through the above polyethylene tube and the solution was administered in sync with the breath of the rat in 1 to 2 seconds. 15.4 μmol /kg (5 mg alendronate/kg) of Alendronate, PEG (2000)-alendronate, and PEG (500)-alendronate was respectively administered to the rat by a pulmonary route. 45 seconds after the administration, the rat was placed at 10° and 250 μl of blood was sampled from the jugular vein in a time-dependent manner. The blood sample was centrifuged (13000 rpm, 10 min) to obtain the plasma fraction and it was stored at −30° C. right before the analysis.

### Measurement of Plasma Ca²⁺ Concentration

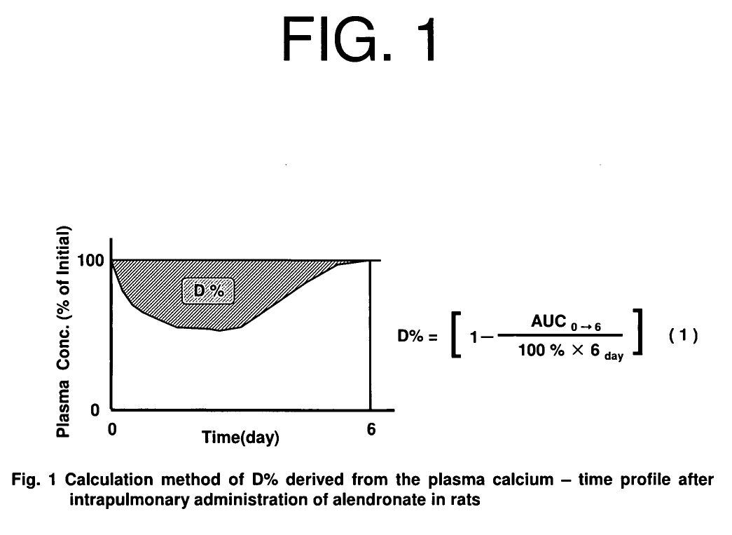

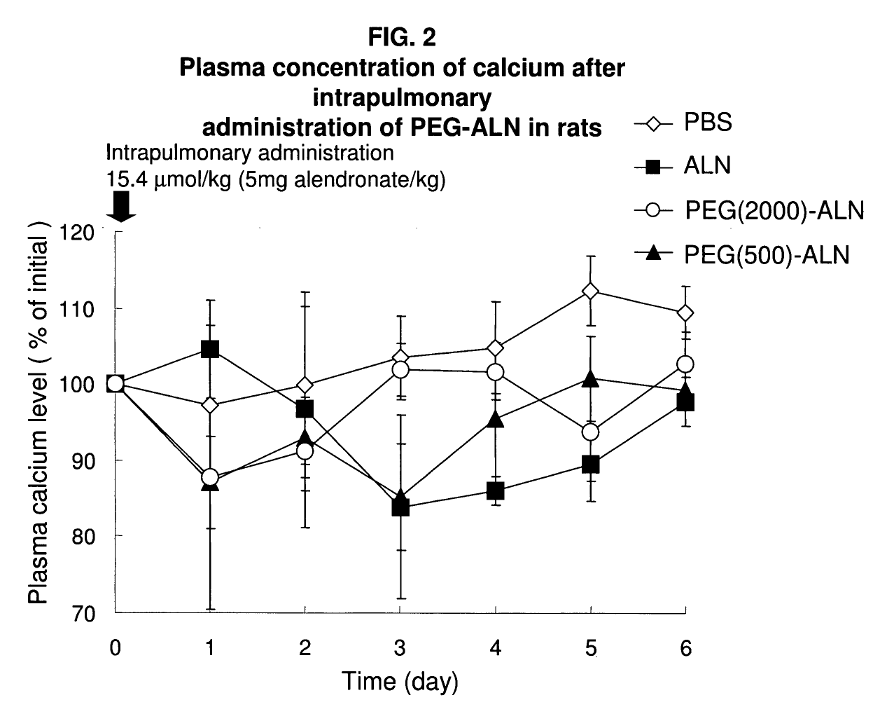

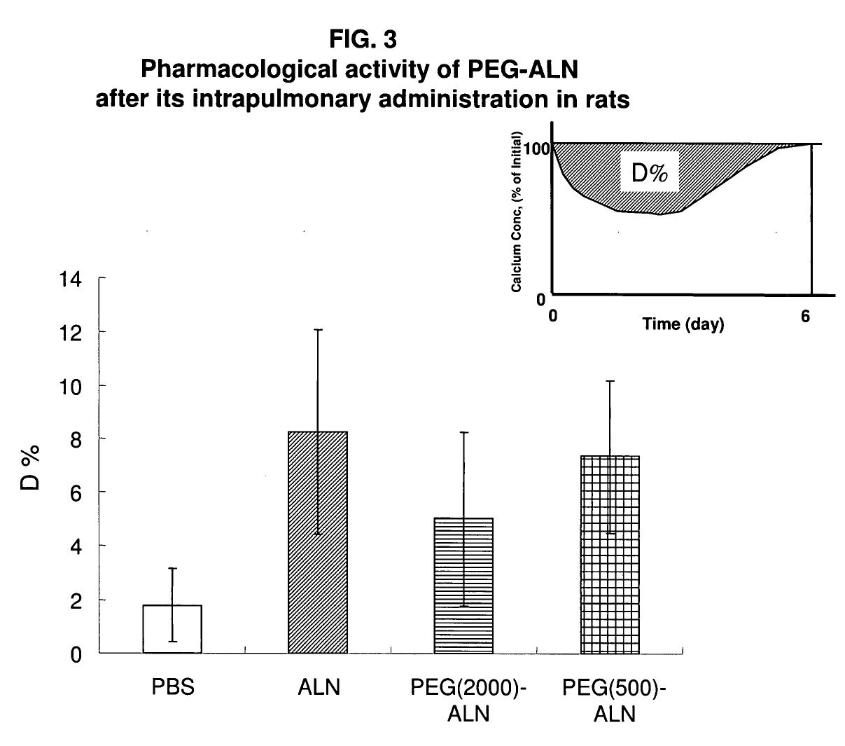

The Ca²⁺ concentration in the plasma obtained was measured by using Calcium E-Test Wako (Wako Pure Chemicals) based on the orthocresolphthalein complexone (OCPC) method. The D % (area above the hypocalcemic effect (%)—time curve ) (See FIG. 1) was also calculated from the Ca²⁺ concentration in the plasma and time curve after administration as an index of the pharmacological effect. The results are shown in FIGS. 2 and 3.

### Evaluation of Intrapulmonary Inflammation

The dosing solution was administered to the rat by a pulmonary route in accordance with the transpulmonary absorption experiment method. In the fourth hour after administration, under pentobarbital anesthesia, the rat was blooded through the main artery and the normal saline solution was poured into the lungs to rinse them. The center of the neck of the rat was cut open to expose the bronchial tract. A polyethylene tube was inserted into the bronchial tract and the bronchoalveolar lavage fluid (BALF) was collected using the PBS 16 ml (4 ml×4). The collected bronchoalveolar lavage fluid (BALF) was centrifuged at 4° C., 200×g, and for 7 minutes. The supernatant fluid was used to measure the LDH activity and the total protein concentration.

### Measurement of LDH Activation

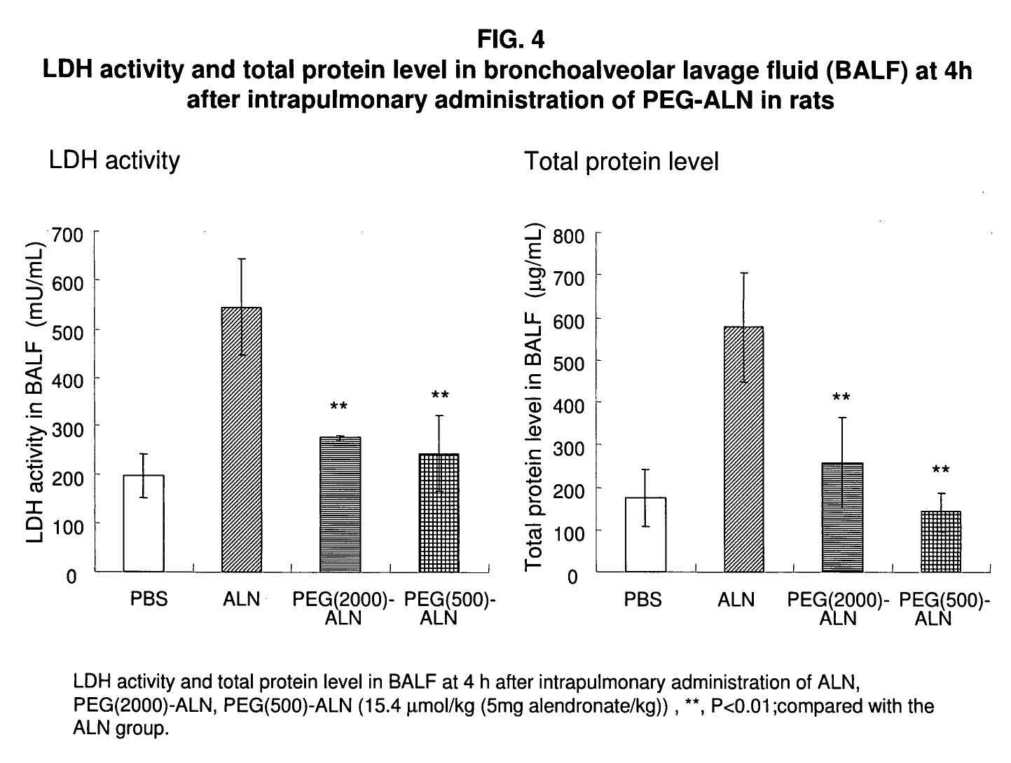

LDH activity is assayed using the LDH-Cytotoxic Test (Wako Pure Chemical Industries, Ltd., Osaka, Japan). LDH is a stable enzyme which is present in all cell types. When the plasma membrane of a cell is damaged, LDH is rapidly released from the cell. Measuring the level of LDH activity in the serum is the most widely used marker in cytotoxicity studies. A high level of LDH activity detected indicates a high degree of irritation, while a low level of LDH activity detected indicates a low degree of irritation. The results are shown in FIG. 4.

### Measurement of Total Protein Concentration

The total protein concentration was measured in the Bradford method. In other words, a color reaction using Coomassie Brilliant Blue was used with bovine serum albumin (BSA) as a reference material. The results are shown in FIG. 4

Composition Method of PEG(2000)-alendronate

500 mg of Alendronate was dissolved in 30 ml of ultrapure water and the pH was adjusted to be 7.0 with 0.2 N NaOH. 180 mg of amino group reactive activation PEG (2000) was added and the pH was adjusted to be 9.0 with 0.2 N NaOH. The mixture was stirred for 2 hours at a room temperature. After alendronate and amino group reactive activation PEG (2000) were reacted, a dialysis was conducted for 24 hours and unreacted alendronate was removed. The PEG (2000)-alendronate solution was freeze-dried to obtain powdered PEG (2000)-alendronate. Part of powdered PEG (2000)-alendronate was dissolved in ultrapure water. It was confirmed that PEG (2000) was combined with the amino group in alendronate by assaying phosphoric acid and the amino group derived from alendronate.

Composition Method of PEG(500)-alendronate

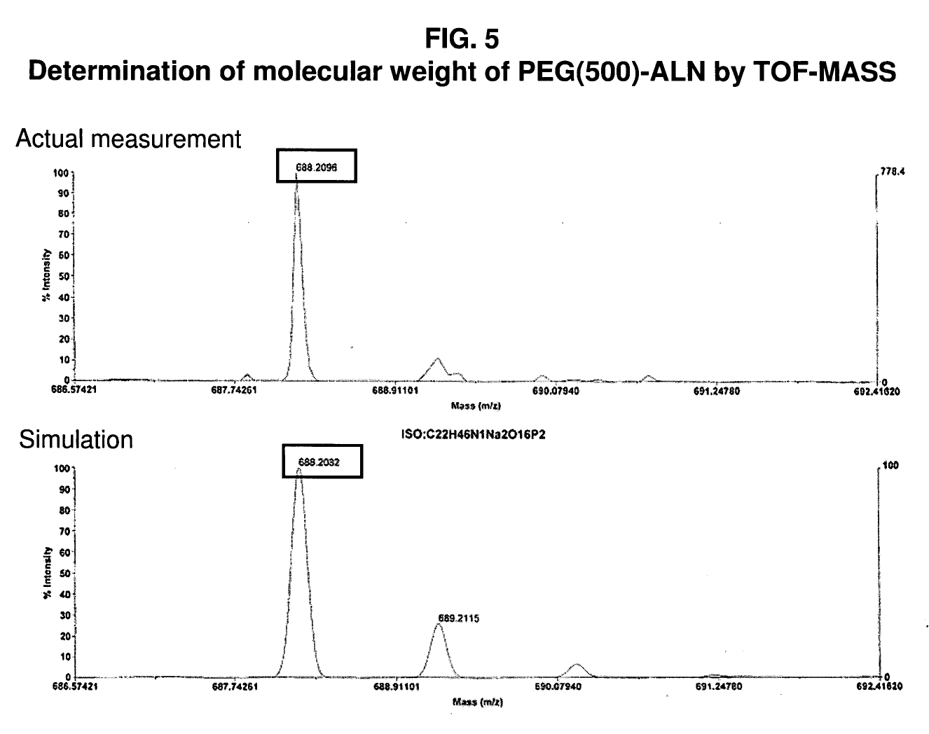

500 mg of Alendronate was dissolved in 30 ml of ultrapure water and the pH was adjusted to be 7.0 with 0.2 N NaOH. 50 mg of amino group reactive activation PEG (500) was added and the pH was adjusted to be 9.0 with 0.2 N NaOH. The mixture was stirred for 2 hours at a room temperature. After alendronate and amino group reactive activation PEG (500) were reacted, it was freeze-dried and rough powder was obtained. Ethanol was added to the rough powder and unreacted alendronate was educed. After it was centrifuged at 1500×g and for 20 minutes, the supernatant fluid was collected. After it was condensed by an evaporator, it was freeze-dried by adding ultrapure water and powdered PEG (500)-alendronate was obtained. Part of powdered PEG (500)-alendronate was dissolved in ultrapure water. It was confirmed that PEG (500) was combined with the amino group in alendronate by assaying phosphoric acid and the amino group derived from alendronate. The molecular weight of PEG (500)-alendronate was measured by TOF-MASS. See FIG. 5.

### Assay of Phosphoric Acid

A color reaction using ammonium molybdate was observed with alendronate as a reference material in the method as described in P.S. Chen, Jr., T. Y. Toribara, and H. Warner. Anal. Chem., 28, 1756 (1956).

### Assay of Amino Group

A color reaction using trinitrobenzenesulfonic acid (TNBS) was observed with alendronate as a reference material in the method as described in A.F. Habeeb. Anal. Biochem., 14, 328-36 (1966).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of administering a bisphosphonate active agent to a subject in need thereof, said method comprising:
administering by a pulmonary route to said subject an effective amount of polymer-linked-bisphosphonate active agent.

2. The method according to claim 1, wherein said polymer-linked-bisphosphonate active agent comprises the structure:
PM-L-BP;
wherein PM is a linear or branched water-soluble and non-peptide polymer having at least one terminus, wherein the terminus is covalently bonded to L; L is a linker; and BP is a bisphosphonate group.

3. The method according to claim 2, wherein said bisphosphonate group is a compound of formula (I): 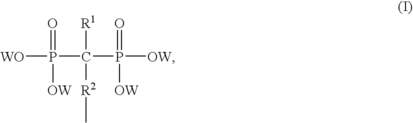
or the pharmaceutically acceptable salts, solvates, hydrates, and prodrug forms thereof, and stereoisomers thereof;
wherein R¹ and R² are independently selected from the group consisting of hydrogen, —OH, halogen, aryl, substituted aryl, pyridyl, furanyl, pyrrolidinyl, imidazonyl, C₁-C₃₀ alkyl, C₁-C₃₀ substituted alkyl, NH₂, NHR³, NR³₂, SH, and SR³, where R³ is C₁-C₃₀ alkyl, C₁-C₁₀ alkoxy, aryl or substituted aryl, and W is selected from the group consisting of hydrogen, alkyl, substituted alkyl, aryl, substituted aryl, Na+, and K+; with the provisio that R² is not a hydrogen, —OH, halogen, NH₂, or SH.

4. The method according to claim 1, wherein said polymer-linked-bisphosphonate active agent is a polymer-linked-alendronate active agent.

5. The method according to claim 1, wherein said polymer-linked-bisphosphonate active agent is a polymer-linked-pamidronate active agent.

6. The method according to claim 2, wherein PM is a polymer selected from the group consisting of poly(alkylene glycol), poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, and copolymers, terpolymers, derivatives and mixtures thereof.

7. The method according to claim 6, wherein PM is a poly(alkylene glycol).

8. The method according to claim 6, wherein PM is a poly(ethylene glycol).

9. The method according to claim 8, wherein PM is PEG(2000).

10. The method according to claim 8, wherein PM is PEG(500)

11. The method according to claim 2, wherein L is a bond, a residue of a functional group used to attach the bisphosphonate group to the polymer, or a C₁-C₄ alkyl comprising one or more hydrolytically stable linkage selected from the group consisting of ester linkages, ether linkages, thio-ether linkages, amide linkages, amine linkages, urea linkages, or carbamate linkages.

12. The method according to claim 11, wherein L is 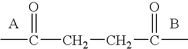
wherein bond A is attached to PM and bond B is attached to BP.

13. The method according to claim 11, wherein L is 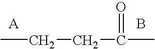
wherein bond A is attached to PM and bond B is attached to BP.

14. The method according to claim 1, wherein said pulmonary route comprises inhalation.

15. The method according to claim 1, wherein said method is of treating said subject for a bone adsorption disease.

16. The method according to claim 15, wherein said subject has been diagnosed as suffering from said bone adsorption disease.

17. The method according to claim 15, wherein said subject has been diagnosed as being at risk for suffering from said bone adsorption disease.

18. The method according to claim 17, wherein said bone adsorption disease is osteoporosis, osteopenia, urolithiasis, hypercalcemia, Paget's disease, bone metastasis, multiple myeloma, or neoplastic bone lesion.

19. A pharmaceutical composition comprising a polymer-linked-bisphosphonate active agent and in a pharmaceutically acceptable vehicle, wherein said pharmaceutical composition is an aerosol.

20. The pharmaceutical composition according to claim 19, wherein said polymer-linked-bisphosphonate active agent comprises the structure:
PM-L-BP;
wherein PM is a linear or branched water-soluble and non-peptide polymer having at least one terminus, wherein the terminus is covalently bonded to L;
L is a linker; and
BP is a bisphosphonate group.

21. The pharmaceutical composition according to claim 20, wherein said bisphosphonate group is a compound of formula (I): 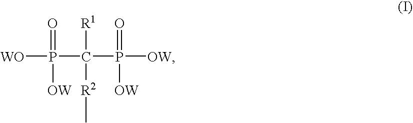
or the pharmaceutically acceptable salts, solvates, hydrates, and prodrug forms thereof, and stereoisomers thereof;
wherein R¹ and R² are independently selected from the group consisting of hydrogen, —OH, halogen, aryl, substituted aryl, pyridyl, furanyl, pyrrolidinyl, imidazonyl, C₁-C₃₀ alkyl, C₁-C₃₀ substituted alkyl, NH₂, NHR³, NR³₂, SH, and SR³, where R³ is C₁-C₃₀ alkyl, C₁-C₁₀ alkoxy, aryl or substituted aryl, and W is selected from the group consisting of hydrogen, alkyl, substituted alkyl, aryl, substituted aryl, Na+, and K+; with the provisio that R² is not a hydrogen, —OH, halogen, NH₂, or SH.

22. The method according to claim 19, wherein said polymer-linked-bisphosphonate active agent is a polymer-linked-alendronate active agent.

23. The method according to claim 19, wherein said polymer-linked-bisphosphonate active agent is a polymer-linked-pamidronate active agent.

24. The pharmaceutical composition according to claim 19, wherein PM is a polymer selected from the group consisting of poly(alkylene glycol), poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, and copolymers, terpolymers, derivatives and mixtures thereof.

25. The pharmaceutical composition according to claim 24, wherein PM is a poly(alkylene glycol).

26. The pharmaceutical composition according to claim 25, wherein PM is a poly(ethylene glycol).

27. The pharmaceutical composition according to claim 26, wherein PM is PEG(2000).

28. The pharmaceutical composition according to claim 26, wherein PM is PEG(500).

29. The pharmaceutical composition according to claim 19, wherein L is a bond, a residue of a functional group used to attach the bisphosphonate group to the polymer, or a C₁-C₄ alkyl comprising one or more hydrolytically stable linkage selected from the group consisting of ester linkages, ether linkages, thio-ether linkages, amide linkages, amine linkages, urea linkages, or carbamate linkages.

30. The pharmaceutical composition according to claim 29, wherein L is 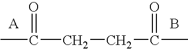
wherein bond A is attached to PM and bond B is attached to BP.

31. The pharmaceutical composition according to claim 29, wherein L is 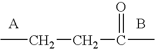
wherein bond A is attached to PM and bond B is attached to BP.

32. The pharmaceutical composition according to claim 19, wherein said aerosol is a liquid aerosol.

33. The pharmaceutical composition according to claim 19, wherein said aerosol is a solid aerosol.

34. The pharmaceutical composition according to claim 33, wherein said solid aerosol comprises a dry powder.

35. The pharmaceutical composition according to claim 34, wherein said powder comprises particles ranging in size from about 1 to about 100 μm.

36. A pharmaceutical composition comprising a polymer-linked-bisphosphonate active agent and in a pharmaceutically acceptable vehicle;
wherein said polymer-linked-bisphosphonate active agent comprises the structure:
PM-L-BP;
wherein PM is a PEG comprising the formula R^{c}—(CH₂CH₂O)ₚ—, where p is from about 3 to about 4000, and R^{c} is a hydrogen, CH₃—O—, CH₂CH₂—O—, CH₃CH₂ CH₂—O— or CH₃—; L is a linker; and BP is a bisphosphonate group.

37. The pharmaceutical composition according to claim 36, wherein said bisphosphonate group is a compound of formula (I): 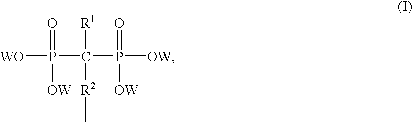
or the pharmaceutically acceptable salts, solvates, hydrates, and prodrug forms thereof, and stereoisomers thereof;
wherein R¹ and R² are independently selected from the group consisting of hydrogen, —OH, halogen, aryl, substituted aryl, pyridyl, furanyl, pyrrolidinyl, imidazonyl, C₁-C₃₀ alkyl, C₁-C₃₀ substituted alkyl, NH₂, NHR³, NR³₂, SH, and SR³, where R³ is C₁-C₃₀ alkyl, C₁-C₁₀ alkoxy, aryl or substituted aryl, and W is selected from the group consisting of hydrogen, alkyl, substituted alkyl, aryl, substituted aryl, Na⁺, and K⁺; with the provisio that R² is not a hydrogen, —OH, halogen, NH₂, or SH.

38. The pharmaceutical composition according to claim 37, wherein L is a bond, a residue of a functional group used to attach the bisphosphonate group to the polymer, or a C₁-C₄ alkyl comprising one or more hydrolytically stable linkage selected from the group consisting of ester linkages, ether linkages, thio-ether linkages, amide linkages, amine linkages, urea linkages, or carbamate linkages.

39. The pharmaceutical composition according to claim 38, wherein L is 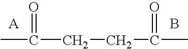
wherein bond A is attached to P and bond B is attached to BP.

40. The pharmaceutical composition according to claim 38, wherein L is 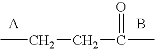
wherein bond A is attached to P and bond B is attached to BP.

41. The pharmaceutical composition according to claim 36, wherein said pharmaceutical composition is an aerosol.

42. The pharmaceutical composition according to claim 41, wherein said aerosol is a liquid aerosol.

43. The pharmaceutical composition according to claim 41, wherein said aerosol is a solid aerosol.

44. The pharmaceutical composition according to claim 43, wherein said solid aerosol comprises a dry powder.

45. The pharmaceutical composition according to claim 44, wherein said powder comprises particles ranging in size from about 1 to about 100 μm.

46. A kit for use in treating a subject suffering from a bone adsorption disease condition, said kit comprising a polymer-linked-bisphosphonate active agent in an inhalable form.

47. The kit according to claim 46, where said kit further comprises a nebulizer, atomizer or inhaler.
